(19) United States
(12) Patent Application Publication
(10) Pub. No.: US 2008/0251070 A1
(43) Pub. Date: Oct. 16, 2008

(54) Method and apparatus for capnography-guided intubation

(76) Inventors: Vadim Pinskiy, Wanaque, NJ (US); Glen Atlas, Livingston, NJ (US); Neil Mori, Fairfield, NJ (US); Harsh Shah, Passaic, NJ (US); Poonam Dudhat, Plainsboro, NJ (US); Vikki Hazel Wood, Wayne, NJ (US)

Correspondence Address: KLAUBER & JACKSON, 411 HACKENSACK AVENUE, HACKENSACK, NJ, 07601, US

(21) Appl. No.: 11/982,711

(22) Filed: Nov. 2, 2007

Related U.S. Application Data
(60) Provisional application No. 60/856,109, filed on Nov. 2, 2006.

(51) Int. Cl. A61M 16/00

(52) U.S. Cl. 128/202.22

(57) ABSTRACT

A method and apparatus for qualitative sensory signal, capnography-guided intubation is provided. A qualitative sensory signal, such as an audible signal, is generated during intubation of a patient to provide an audible indication of carbon dioxide levels, so as to facilitate proper placement of an intubation tube. The frequency of the audible signal corresponds to measured carbon dioxide levels, thereby providing a simple, easy-to-interpret, audible indication of the current position of an endotracheal tube during intubation, as well as confirmation of proper placement of the tube. Alternatively, the qualitative sensor signal may be an omni-directional visual signal or a palpable vibratory signal.

### RELATED APPLICATIONS

The present application claims the benefit under 35 U.S.C. § 119 of U.S. Provisional Application No. 60/856,109, filed Nov. 2, 2006, the contents of which is hereby incorporated by reference in its entirety.

### BACKGROUND OF THE INVENTION

1. Field of the Invention

The present invention relates to a device for ensuring proper intubation of a patent. More specifically, the present invention relates to a method and apparatus for capnography-guided intubation, where an auxiliary sensory indicator is included to assist therewith.

2. Related Art

Intubation is an important medical procedure for ensuring that a patient with a blocked airway is able to breath. Frequently, intubation is practiced by inserting an endotracheal tube into the trachea of a patient, often with the assistance of a laryngoscope. Once inserted, the endotracheal tube can be connected to life support equipment, such as a respirator, to provide oxygen to the patient. In orotracheal intubation, the endotracheal tube is inserted into the mouth of the patient, guided past the larynx, and placed into the trachea. In nasotracheal intubation, a tube is inserted through a nostril of a patient and guided down to the trachea for placement therein. Still further, tracheal intubation involves a surgical incision into the trachea (tracheotomy), followed by insertion of a tube into the incision and placement of the tube in the trachea.

In each of the foregoing types of intubation, it can be difficult to properly guide the tube into the trachea of the patient, due to anatomical variations of the patient or abnormalities of larynx, trachea, or surrounding structures. Further, proper placement of the tube can be difficult due to injuries or diseases, such as gunshot wounds or tumors. Moreover, there always exist the dangers that vital anatomical structures can be damaged during the intubation process, or that the tube can be inadvertently inserted into the esophagus rather than the trachea. As such, there is a need to guide the intubation process to avoid these dangers.

Capnography is a known technique for use in the intubation process. Using capnography, carbon dioxide levels can be measured during intubation. Such levels provide an accurate indication of whether the endotracheal tube is properly guided into the patient's trachea. Low carbon dioxide levels (<5 mmHg) provide an indication that the endotracheal tube is not properly placed into the patient's trachea. Conversely, high carbon dioxide levels (>5 mmHg) provide an indication that the endotracheal tube is properly placed into the trachea.

A particular problem with existing, capnography-guided intubation techniques is that the user is not provided with a simple, easy to interpret indication of carbon dioxide levels, in real time. For example, while a capnograph can provide a visual indication (e.g., on a display panel) of carbon dioxide levels, the operator is forced to draw his or her attention away from the intubation process to view and interpret the carbon dioxide readings. This increases the danger of hurting the patient and prolonging the intubation process. Moreover, it is inconvenient and impractical to review numerical indications of carbon dioxide levels and to interpret same to determine whether intubation is occurring properly. Still further, in most intubation applications, capnographs are only used to confirm proper intubation after the procedure is complete. In such circumstances, if the intubation is not proper, the entire endotracheal tube must be removed and the intubation process repeated, thus increasing the possibility of injury to the patent as well as initiating asphyxia, which could cause brain injury or death if the intubation is not quickly corrected.

Accordingly, what would be desirable, but has not yet been provided, is a method and apparatus for capnography-guided intubation which solves the foregoing shortcomings of existing intubation techniques.

### SUMMARY OF THE INVENTION

The present invention relates to a method and apparatus for capnography-guided intubation. The apparatus includes a conventional endotracheal tube or stylet for intubating a patient, a carbon dioxide sensor for measuring carbon dioxide levels during intubation of the patient, a vacuum pump connected to the endotracheal tube or stylet for drawing air from the patient's lungs and past the carbon dioxide sensor, a capnograph connected to the carbon dioxide sensor for measuring carbon dioxide levels, and an auxiliary sensor for the transmission and reception of a qualitative signal, such as a flashing light, a vibration signal or an audible signal indicative of carbon dioxide levels, in real time, during intubation of the patient. In a particular embodiment, the auxiliary sensor may comprise an audio circuit and associated audio output device for generating such audible signal. The auxiliary sensor provides an instant, easy-to-interpret, qualitative indication of carbon dioxide levels to guide the intubation process and to confirm proper placement of the endotracheal tube into the trachea of the patient. The apparatus could be provided in a single, portable housing that can be easily transported to a patient's location. Alternatively, all or a portion of the components of the apparatus could be provided in a handheld unit. For example, a handheld device could include an integral vacuum pump and carbon dioxide sensors, and could be connected to an endotracheal tube or stylet as well as to an external capnograph and an alarm, that may be visually omnidirectional, vibratory or audible. A graphical user interface is provided for displaying real-time carbon dioxide levels measured by the present invention, as well as for storing patient-related information.

Accordingly as a first aspect of the present invention, there is a system for determining the location of a intubation device in a patient, the system comprising a vacuum system for withdrawing gas from a patient through an intubation device, a sensing system for sensing the level of carbon dioxide in the withdrawn gas by the vacuum system and to provide a signal indicative of the level of carbon dioxide, and an auxiliary sensor for transmitting a qualitative sensory signal that is indicative of the level of carbon dioxide in the gas withdrawn by the vacuum system. The qualitative sensory signal may comprise an omnidirectional light signal such as a flashing light, a palpable vibratory signal, or an audio signal. In the instance of the last mentioned signal, an audio system can be included that is adapted to convert the signal from the sensing system into the audible sound.

In a further aspect, the system includes a carbon dioxide sensor located proximate to an intubation device that produces a signal indicative of the level of carbon dioxide in the withdrawn gas and in another aspect, the system includes a capnograph adapted to receive the signal from the carbon dioxide sensor and to produce a voltage signal indicative of the level of carbon dioxide in the withdrawn gas.

In a still further aspect, the invention includes an auxiliary processer unit such as an audio circuit, that receives the voltage signal from the capnograph and converts that voltage signal to a qualitative signal or alarm, such as a vibration, a flashing light, or an audible sound, any or all of which may vary in frequency in accordance with the level of the voltage signal. Still another aspect is a vacuum system that comprises a vacuum pump.

As another aspect of the present invention, there is a handheld device for determining the intubation of a patient comprising an inlet and an outlet, and a duct communicating between the inlet and the outlet, a vacuum pump connected to the duct for drawing gas through the duct, a carbon dioxide sensor located proximate to the duct for monitoring the level of carbon dioxide in the gas passing through the duct and for providing a signal indicative of that sensed level of carbon dioxide. A still further aspect includes a valve for controlling the flow of gas through the outlet. As another aspect, the carbon dioxide sensor is an infrared sensor. Further yet, the inlet of the device comprises an adapter for connection to an intubation device. As a still further aspect, the device comprises an adapter for connection to an endotracheal tube.

In another aspect of the present invention, there is a system for determining the location of an intubation device within a patient comprising an intubation device having a distal end and a proximal end, a system for withdrawing gas through the intubation device from a location within the patient proximate to the distal end of the intubation device, a sensing system for sensing the level of carbon dioxide in the gas passing through the intubation device and providing a signal indicative of the level of the sensed carbon dioxide, the sensing system including an auxiliary sensor, such as an audio system, that receives the signal from the sensing system and converts that signal into a qualitative sensory signal such as an omni-directional light signal (e.g. a flashing light), a vibratory signal, or an audio signal indicative of the level of carbon dioxide in the gas withdrawn by the vacuum system. In a still further aspect, the system includes a vacuum pump. In another aspect, the intubation device is an endotracheal tube. Still further, the sensing system of the invention provides a voltage having a magnitude indicative of the level of sensed carbon dioxide and in the case of the audible sound signal, includes an audio circuit and audio output device that converts the voltage signal into such audible sound, that is indicative of the level of sensed carbon dioxide. Yet further, the audible sound has a frequency that is indicative of the level of sensed carbon dioxide.

In a still further aspect of the present invention there is a method of determining the location of an intubation device in a patient comprising the steps of;

(a) introducing an intubation device having a distal end and a proximal end into a patient,

(b) removing a sample of gas from the patient at a location proximate to the distal end of the intubation device;

(c) determining the carbon dioxide content of the sample of gas removed from the patient; and

(d) creating a qualitative sensory signal selected for the group consisting of an omnidirectional light signal, a vibratory signal or an audio signal indicative of the level of carbon dioxide determined in step (c).

In this aspect, and with respect to the audible signal, there further is included the step of creating an audible sound that comprises providing an electrical voltage that is indicative of the level of carbon dioxide determined in step (c) and converting the electrical voltage to an audible sound having a frequency indicative of the level of carbon dioxide determined in step (c).

In a further aspect, the step of introducing an intubation device into a patient includes the step of repositioning the intubation device based on the sensory signal created in step (d).

Still further there is an aspect wherein the step of determining the carbon dioxide content of the sample of gas removed from the patient comprises positioning a carbon dioxide sensor proximate to the proximal end of the intubation device.

Finally, as a still further aspect, the method includes the step of removing a sample of gas from the patient at a location proximate to the distal end of the intubation device comprises applying a vacuum to the intubation device to draw gas from the distal end of the intubation device.

Accordingly, it is a principal object of the present invention to provide a method for the capnography-assisted intubation of a patient which simplifies the management of the process and thereby reduces the possibility of error and harm to the patient.

It is a further object of the present invention to provide a system for use in the method as aforesaid, that introduces a sensory signal, such as an audible sound, as an indication of patient carbon dioxide level that does not require the diversion of attention from the intubation procedure.

It is a still further object of the invention to provide a method and system as aforesaid, where the sensory signal provides the indication in real time.

It is yet a further object of the invention to provide a system as aforesaid, which may comprise a hand held device for use in the present method.

Other objects and advantages will become apparent to those skilled in the art from a consideration of the ensuing detailed description taken in conjunction with the following illustrative drawings.

### BRIEF DESCRIPTION OF THE DRAWINGS

FIG. 1 is a diagram showing an audible, capnography-guided intubation apparatus constructed in accordance with a particular alternate embodiment of the present invention;

FIG. 2 is a flowchart showing a process for operating the apparatus of FIG. 1;

FIG. 3 is a block diagram showing the audio circuit of the present invention in greater detail;

FIG. 4 is an electrical schematic of the audio circuit of the present invention;

FIG. 5 is a diagram showing a portable, handheld device according to the present invention for capnography-guided intubation; and

FIG. 6 is a screenshot of a graphical user interface screen according to the present invention for displaying real-time carbon dioxide levels and for storing and displaying patient-related information.

### DETAILED DESCRIPTION OF THE INVENTION

The present invention relates to a method and apparatus for capnography-guided intubation. The present invention is operable with any suitable endotracheal tube or stylet, and includes a carbon dioxide sensor, a vacuum source for drawing air from the patient's lungs and past the carbon dioxide sensor, a capnograph, and an auxiliary sensor such as an audio circuit and associated audio output device. A sensory signal, such as an omnidirectional visual display or light, a vibratory signal, or an audible signal, is generated during intubation of a patient to provide an audible indication of carbon dioxide levels, so as to facilitate proper placement of an intubation tube. In the case of the audible signal, the frequency (pitch) corresponds to measured carbon dioxide levels, thereby providing a simple, easy-to-interpret, indication of the current position of an endotracheal tube during intubation, as well as confirmation of proper placement of the tube.

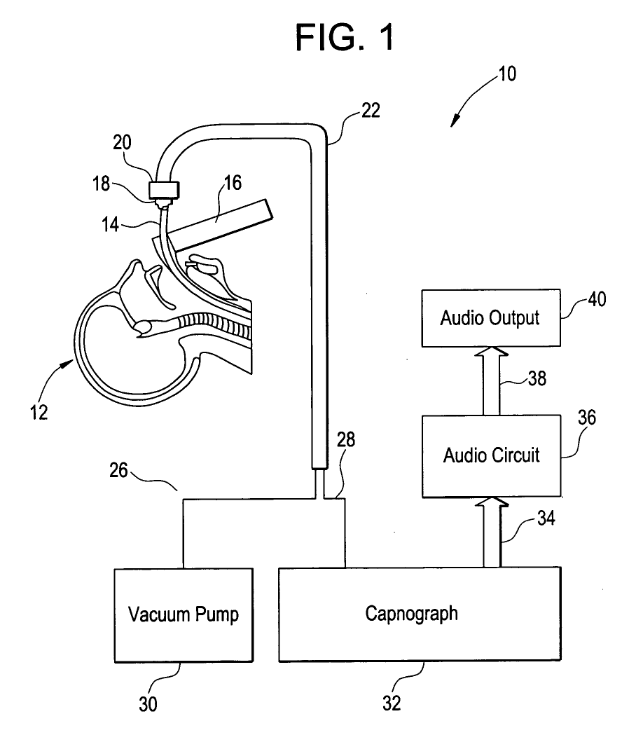

FIG. 1 is a diagram showing an apparatus 10 constructed in accordance with the present invention, for providing capnography-guided intubation of a human 12. The apparatus 10 is operable with any conventional endotracheal tube, such as the endotracheal tube 14, or a suitable stylet (hollow or solid). It has been found that endotracheal tubes with sizes of 7 to 9 mm are suitable, such as those sold by Hudson RCI, Inc. As is known in the art, a laryngoscope 16 can be used to facilitate insertion of the endotracheal tube 14 into the airway of the human 12. The apparatus 10 includes an adapter 18 for allowing removable connection of the endotracheal tube 14 or a stylette thereto, a carbon dioxide (CO₂) sensor 20, a reinforced tube 22 having an air tube 26 and an electrical cable 28 extending therethrough, a vacuum pump 30, a capnograph 32, and an auxiliary sensor means, illustrated by audio circuit 36, that is connected to an audio output device 40 (e.g., a speaker). The air tube 26 establishes fluid communication between the endotracheal tube 14 and the vacuum pump 30, and the electrical cable 28 establishes electrical communication between the sensor 20 and the capnograph 32. A trap could be provided before the vacuum pump 30 to prevent fluids from the airway of the human 12 from damaging the pump 30.

During intubation of the human 12, the vacuum pump 30 is operated to draw air from the lungs of the human 12, through the endotracheal tube 14, and past the sensor 20, so that carbon dioxide levels in the air can be measured by the sensor 20. The vacuum pump 30 could include the B-series micro air pump (Model No. BP120CNNN) manufactured by Sensidyne, Inc., or any other suitable vacuum pump. The sensor 20 converts carbon dioxide levels into electrical signals which are processed by the capnograph 32. It should be noted that the sensor 22 could be an infrared carbon dioxide sensor, or any other suitable type of sensor. The sensor 20 and the capnograph 32 could together comprise the CO₂SMO® Mainstream Capnograph/Pulse Oximeter manufactured by Respironics, Inc., or any other suitable capnography equipment. The capnograph 32 generates an electrical signal 34 corresponding to the level of carbon dioxide sensed by the sensor 20. The voltage level of the signal 34 varies based upon the level of carbon dioxide sensed by the sensor 20.

In the illustrated embodiment, the signal 34 is processed by an audio circuit 36, generating an audio signal 38 that can be sent to an audio output device 40, such as a speaker. The audio circuit 36 converts changes in the voltage level of the signal 34 to changes in the frequency (pitch) of the audio signal 38. An increase in the pitch of the audio signal 38 indicates an increase in the concentration of carbon dioxide, thereby providing an indication to the operator that the endotracheal tube 14 has entered into the trachea 12 of the human, or is properly placed therein. As indicated however, other sensor means and assemblies, not illustrated herein, could be used to perform the same function. Thus, for example, signal 34 could be directed to a circuit that would activate an omni-directional light source, that by its illumination, would provide a like indication of modulation in carbon dioxide levels. Similarly, the output signal could activate a generator of vibratory energy that would provide a like indication to the medical personnel and would thereby warn of fluctuations in carbon dioxide levels. As the invention contemplates and extends to other sensory signal means and corresponding systems, all such variations are intended to be embraced herein, and are considered within the scope hereof, so that the detailed discussion of the audio circuit is illustrative and not restrictive of the claimed invention.

It should be noted that the vacuum pump 30, the capnograph 32, the audio circuit 36, and the audio output device 40 could be provided in a single, portable enclosure that can be easily transported to a patient's bedside, to an operating room, or to any other desired location. Further, the present invention could be maintained in an ambulance or rescue vehicle, and easily transported to an emergency location. Additionally, the apparatus 10 could be battery-powered, or a standard, 120 volt alternating current (AC) power supply could be utilized. Any desired configuration of the components of the apparatus 10 could be provided without departing from the spirit or scope of the present invention.

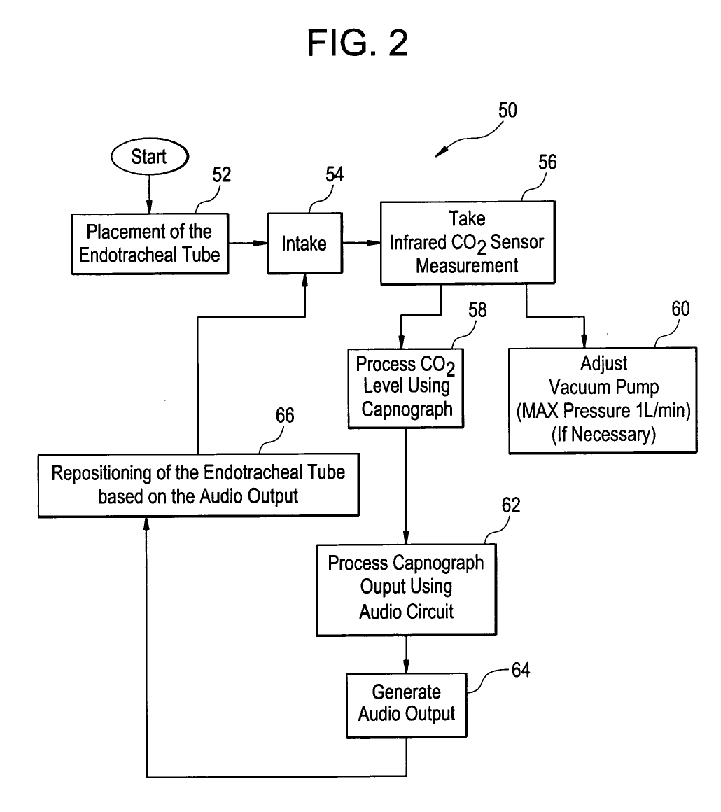

FIG. 2 is a flowchart of a process, indicated generally at 50, for operating the apparatus 10 of FIG. 1. Beginning in step 52, the endotracheal tube 14 of FIG. 1 is placed into the mouth of a patient, and intubation is initiated. In step 54, the vacuum pump 30 of FIG. 1 is operated to draw (intake) air from the patient's lungs into the endotracheal tube and past the carbon dioxide sensor 20 of FIG. 1. In step 56, a carbon dioxide measurement is taken by the sensor 20, and in step 58, the measurement is processed by the capnograph 32 of FIG. 1 to generate an electrical signal corresponding to measured carbon dioxide levels. Optionally, in step 60, the vacuum pump 30 of FIG. 1 could be adjusted to provide a desired airflow rate (e.g., up to a maximum of 1 liter per minute). In step 62, the output (electrical signal) generated by the capnograph 62 is processed by the audio circuit 36 of FIG. 1.

In step 64, an audio signal is generated by the audio circuit 36 of FIG. 1, based upon the electrical signal generated by the capnograph. During initial intubation of the patient (e.g., after inserting the endotracheal tube into the mouth of the patient), carbon dioxide levels are low. As a result, the frequency of the audio signal will be low, thereby indicating to the user that the endotracheal tube 14 of FIG. 1 is not positioned within the airway of the patient. In step 66, the endotracheal tube 14 of FIG. 1 could be repositioned as desired, based upon the frequency of the audio signal. The process 50 is repeated during intubation of the patient, thereby providing an audible indication of carbon dioxide levels in real time. As the endotracheal tube of FIG. 1 is inserted into the trachea, carbon dioxide levels rise, thereby resulting in an increase in the frequency of the audio signal generated by the present invention. Such an increase provides an audible indication to the operator that the endotracheal tube is being properly inserted into the trachea, or confirmation that intubation is complete.

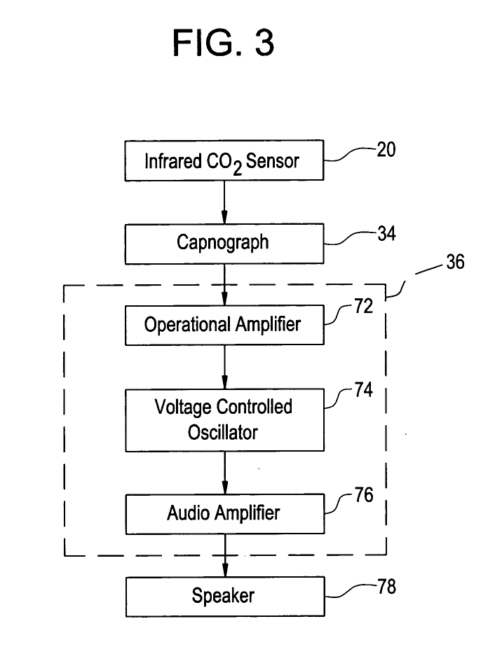

FIG. 3 is a block diagram showing the audio circuit 36 of FIG. 1 in greater detail. As mentioned earlier, the sensor 20 is connected to the capnograph 34 to generate an electrical signal indicative of sensed carbon dioxide levels in real time during intubation of the patient. The audio circuit 36 converts this electrical signal into an audio signal, which provides an audible indication of carbon dioxide levels. The audio circuit 36 includes an operation amplifier 72 for amplifying the electrical signal generated by the capnograph 34, a voltage-controlled oscillator (VCO) 74 for generating an audible signal having a frequency that is adjustable based upon the voltage of the electrical signal amplified by the operational amplifier 72, and an audio amplifier 76 for amplifying the audio signal. The audio signal can drive a speaker 78, or it can be transmitted to another device.

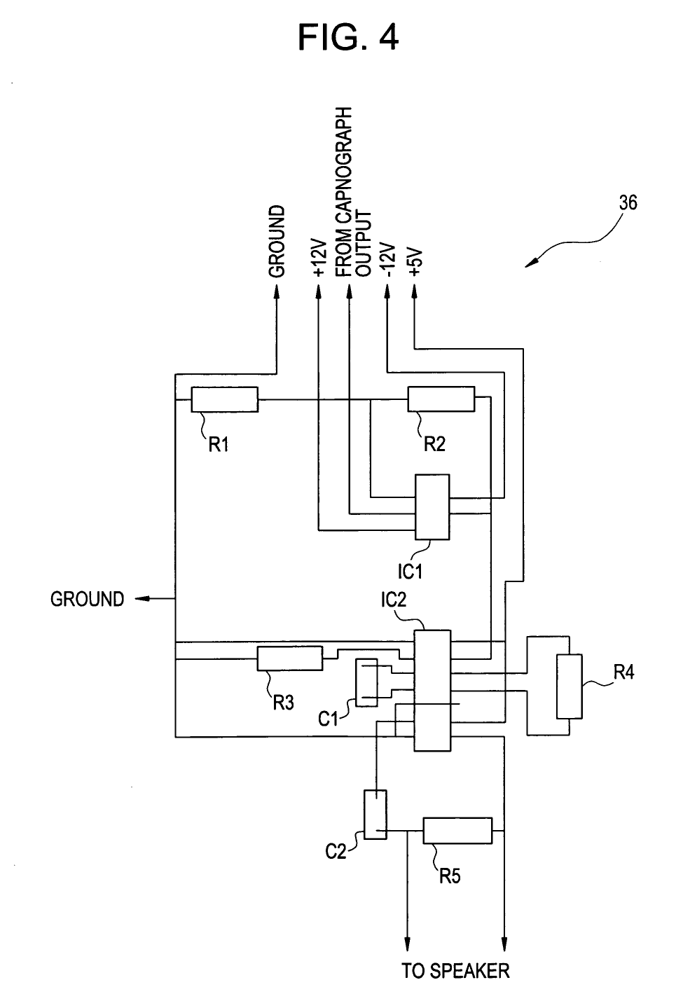

FIG. 4 is an electrical schematic of the audio circuit 36 of FIG. 1. The circuit 36 is connected to electrical ground, direct current (DC) sources of +12 V, −12 V, and +5V, and the electrical output of the capnograph 32 of FIG. 1. The circuit 36 can also be connected to drive a speaker or suitable type of audio output device. The circuit 36 includes integrated circuit IC1, which corresponds to the operational amplifier 72 of FIG. 3. IC1 could include the LM741CN operational amplifier manufactured by National Semiconductor, Inc., or any other suitable operational amplifier. The circuit 36 also includes integrated circuit IC2, which corresponds to the VCO 74 of FIG. 3. IC2 could include the SN54LS624 VCO manufactured by Texas Instruments, Inc., or any other suitable VCO. A plurality of discrete components is also included in the circuit 36, including resistors R1-R5 and capacitors C1-C2. The associated values for these components are given in the following table:

TABLE 1

| Component | Value | |
|---|---|---|
| R1 | 5,000 | Ohms |
| R2 | 1,000 | Ohms |
| R3 | 5,000 | Ohms |
| R4 | 500 | Ohms |
| R5 | 5,000 | Ohms |
| C1 | 47 | microFarads |
| C2 | 100 | microFarads |

It should be noted that the capacitor C2 and the resistor R5 are not required to operate the circuit 36, but are useful for filtering the audio signal. The circuit 36 can generate an audio signal in the audible range of 20 Hz to 20 kHz. As indicated previously, low audio frequencies correspond to low carbon dioxide levels (indicating that the endotracheal tube is not in the patient's airway) and high audio frequencies correspond to high carbon dioxide levels (indicating proper placement of the endotracheal tube in the patient's airway).

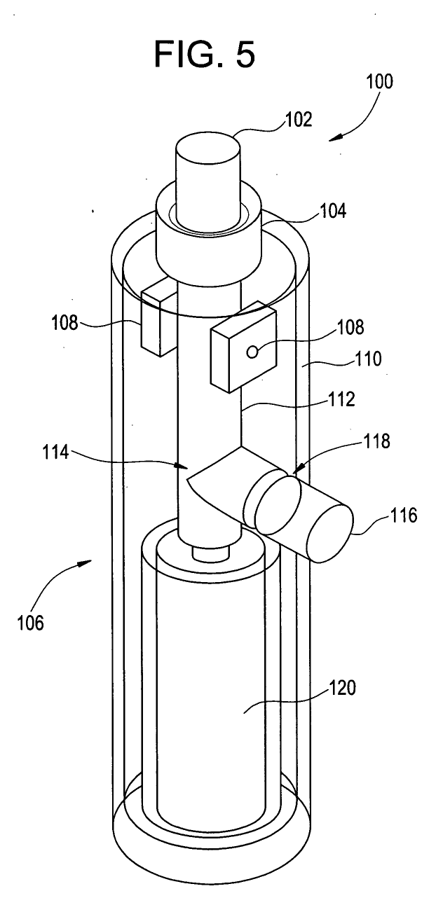

FIG. 5 is a perspective view of a handheld device 100 according to the present invention for monitoring carbon dioxide levels during intubation. The device 100 includes an adaptor 104 for allowing removable attachment of input tubing 102 (e.g., an endotracheal tube or a stylette) and a handheld portion 106 including a housing 110, a plurality of infrared carbon dioxide detectors 108, a duct 112, a valve 114, an exhaust tube 116 extending through an aperture 118 in the housing 110, and a vacuum pump 120. During intubation, the valve 114 (which could be manually-operated or electronic) and the vacuum pump 120 are operated to draw air through the tubing 102, past the infrared detectors 108, through the duct 112, and to vent same through the exhaust tubing 116. The infrared detectors 108 monitor carbon dioxide levels during intubation, in the manner described herein, and are in electrical communication with a capnograph and the audio circuit of the present invention to provide an audible signal indicative of carbon dioxide levels. It should be noted that the device 100 could also include an internal battery for powering same, or it could be connected to an external (e.g., 120 volt AC) power supply.

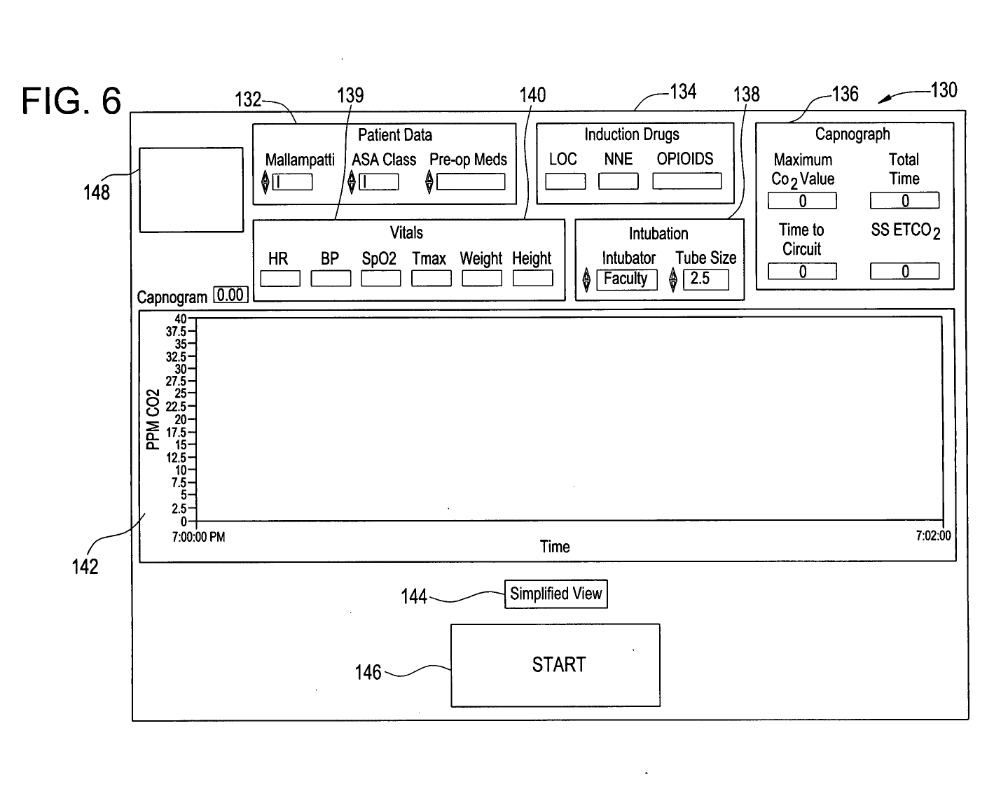

FIG. 6 is a screenshot showing a graphical user interface 130 according to the present invention for displaying patient data and carbon dioxide levels during intubation of a patient. The interface 130 could execute on any suitable computer system connected to the capnograph of the present invention, such as a personal computer, a workstation, or a portable computing device (e.g., a personal digital assistant (PDA), pocket computer, or the like). The interface 130 could be coded using any suitable programming language known in the art, such as Java or C++. Further, the interface 130 could be linked to a relational database management system (DBMS) for storing and accessing patient-related data.

The interface 130 includes data fields 132 for entering patient data. Data fields 134 allow for the entry of information relating to induction drugs. Information about the capnograph could be entered in fields 136, and intubation information can be entered in fields 138. Vital statistics about the patient could be entered in fields 139. A graph area 142 displays real-time carbon dioxide levels measured during intubation. A button 144 can be clicked by a user to display a simplified view, i.e., a view containing less information than described above. Start and stop buttons 146 and 148 can be clicked as desired to start and stop the capturing of carbon dioxide level information.

As stated above in respect to the sensory indicator component of the invention, it should be noted that the present invention could be modified to provide other types of indications of carbon dioxide levels, in real time, such as visual, tactile, or other indications. For example, a light could be provided and modulated (e.g., flashed at different rates) to indicated various carbon dioxide levels, or an array of lights (e.g., a light-emitting diode (LED) array) could be provided for displaying such levels. Moreover, any suitable type of display, such as an LED display, liquid crystal display (LCD), flat panel display, cathode ray tube (CRT), or other types of displays, could be used to indicate carbon dioxide levels. Other types of indicators, such as tactile signals, could also be implemented. For example, the present invention could include a vibration source which vibrates across a span of frequencies to indicate various carbon dioxide levels. Thus, as will be readily appreciated, the present invention could be modified to provide any desired type of sensory indication of carbon dioxide levels.

Having thus described the invention in detail, it is to be understood that the foregoing description is not intended to limit the spirit or scope thereof.

What is claimed is:

1. A system for determining the location of a intubation device in a patient, the system comprising: a vacuum system for withdrawing gas from a patient through an intubation device, a sensing system for sensing the level of carbon dioxide in the withdrawn gas by the vacuum system and to provide a signal indicative of the level of carbon dioxide, an auxiliary sensor adapted to convert the signal from the sensing system into a qualitative sensory signal selected from the group consisting of an omnidirectional light signal, a vibratory signal or an audio signal indicative of the level of carbon dioxide in the gas withdrawn by the vacuum system.

2. The system as defined in claim 1 wherein the sensing system includes a carbon dioxide sensor located proximate to an intubation device that produces a signal indicative of the level of carbon dioxide in the withdrawn gas.

3. The system of claim 2 wherein the sensing system further includes a capnograph adapted to receive the signal from the carbon dioxide sensor and to produce a voltage signal indicative of the level of carbon dioxide in the withdrawn gas.

4. The system of claim 3 wherein the auxiliary sensor includes an audio circuit that receives the voltage signal from the capnograph and converts that voltage signal to an audible sound that varies in frequency in accordance with the level of the voltage signal.

5. The system of claim 3 wherein the vacuum system comprises a vacuum pump.

6. A handheld device for determining the intubation of a patient comprising an inlet and an outlet, and a duct communicating between the inlet and the outlet, a vacuum pump connected to the duct for drawing gas through the duct, a carbon dioxide sensor located proximate to the duct for monitoring the level of carbon dioxide in the gas passing through the duct and for providing a signal indicative of that sensed level of carbon dioxide.

7. The handheld device as defined in claim 6 wherein the device further includes a valve for controlling the flow of gas through the outlet.

8. The handheld device as defined in claim 6 wherein the carbon dioxide sensor is an infrared sensor.

9. The hand held device as defined in claim 6 wherein the inlet comprises an adapter for connection to an intubation device.

10. The hand held device as defined in claim 6 wherein the inlet comprises an adapter for connection to an endotracheal tube.

11. A system for determining the location of an intubation device within a patient comprising;
an intubation device having a distal end and a proximal end;
a system for withdrawing gas through the intubation device from a location within the patient proximate to the distal end of the intubation device,
a sensing system for sensing the level of carbon dioxide in the gas passing through the intubation device and providing a signal indicative of the level of the sensed carbon dioxide;
an auxiliary sensor receiving the signal from the sensing system and converting that signal into a qualitative sensory signal selected from the group consisting of an omnidirectional light signal, a vibratory signal or an audio signal indicative of the level of carbon dioxide in the gas withdrawn by the vacuum system.

12. The system of claim 11 wherein the system for withdrawing gas comprises a vacuum system using a vacuum pump.

13. The system of claim 11 wherein the intubation device is an endotracheal tube.

14. The system of claim 11 wherein the sensing system provides a voltage having a magnitude indicative of the level of sensed carbon dioxide and the auxiliary sensor includes an audio circuit and audio output device that converts the voltage signal into an audible sound that is indicative of the level of sensed carbon dioxide.

15. The system of claim 14 wherein the audible sound has a frequency that is indicative of the level of sensed carbon dioxide.

16. A method of determining the location of an intubation device in a patient comprising the steps of;
(a) introducing an intubation device having a distal end and a proximal end into a patient,
(b) removing a sample of gas from the patient at a location proximate to the distal end of the intubation device;
(c) determining the carbon dioxide content of the sample of gas removed from the patient; and
(d) creating a qualitative sensory signal selected from the group consisting of an omnidirectional light signal, a vibratory signal or an audio signal indicative of the level of carbon dioxide determined in step (c).

17. The method of claim 16 wherein the step of creating a qualitative sensory signal comprises providing an electrical voltage that is indicative of the level of carbon dioxide determined in step (c) and converting the electrical voltage to an audible sound having a frequency indicative of the level of carbon dioxide determined in step (c).

18. The method of claim 16 wherein the step of introducing an intubation device into a patient includes the step of repositioning the intubation device based on the qualitative sensor signal created in step (d).

19. The method of claim 16 wherein the step of determining the carbon dioxide content of the sample of gas removed from the patient comprises positioning a carbon dioxide sensor proximate to the proximal end of the intubation device.

20. The method of claim 16 wherein the step of removing a sample of gas from the patient at a location proximate to the distal end of the intubation device comprises applying a vacuum to the intubation device to draw gas from the distal end of the intubation device.
